(19) United States
(12) Patent Application Publication
(10) Pub. No.: US 2024/0254323 A1
(43) Pub. Date: Aug. 1, 2024

(54) MIXTURES OF POLYSACCHARIDES AND POLYAMINOSACCHARIDES WITH IMPROVED RHEOLOGICAL PROPERTIES

(71) Applicant: JOINTHERAPEUTICS S.R.L., Como (IT)

(72) Inventors: Lanfranco Callegaro, Como (IT); Giulio Bianchini, Como (IT)

(73) Assignee: JOINTHERAPEUTICS S.R.L., Como (IT)

(21) Appl. No.: 18/560,254

(22) PCT Filed: May 10, 2022
(86) PCT No.: PCT/IB2022/054315

§ 371 Date: Nov. 10, 2023

(30) Foreign Application Priority Data
May 18, 2021 (IT) 102021000012737

(51) Int. Cl. C08L 5/08; A61L 27/20; A61L 27/54; C08B 37/08; C12N 5/00

(52) U.S. Cl. CPC C08L 5/08; A61L 27/20; A61L 27/54; C08B 37/003; C08B 37/0072; C12N 5/0068; C08L 2203/02; C08L 2312/00

(57) ABSTRACT

The present invention concerns a polymeric composition with improved rheological properties, comprising at least one polyanionic polysaccharide, or a derivative thereof, and a polyaminosaccharide chosen from chitosan, functionalised chitosan and salts thereof. The invention furthermore concerns methods for the preparation of said polymeric composition and uses thereof.

### FIELD OF THE INVENTION

The present invention concerns a polymeric composition with improved rheological properties, comprising at least one crosslinked polyanionic polysaccharide, or a crosslinked derivative thereof, and at least one polyaminosaccharide selected from chitosan, functionalised chitosan, and salts thereof. The invention furthermore concerns methods for the preparation of said polymeric composition and uses thereof.

### Background Art

It is known that polysaccharides are biopolymers of great application interest due to their high biocompatibility and due to their peculiar physical/chemical proprieties and in particular due to the viscous behaviour of their aqueous solutions.

Among these biopolymers, hyaluronic acid is undoubtedly among the most interesting from an application point of view and therefore among the most widely utilised in the biomedical field, the solutions thereof having a peculiar rheological behaviour, i.e. featuring interesting viscosity and visco-elasticity characteristics.

Due to its aforesaid characteristics (i.e. viscosity and viscoelasticity), hyaluronic acid has found broad use in both the cosmetic and pharmaceutical fields, both as is and as a drug delivery system. The preferred formulation for the application of hyaluronic acid is that of injectable solutions; think, for example of the injection of hyaluronic acid by intra-articular route to restore the mechanical function of a joint or the injection of hyaluronic acid as a filler in the dermatological and cosmetic fields.

Other polysaccharides also find broad use for the viscous properties of their solutions/dispersions in water. Among those most used both in the pharmaceutical industry and in the food industry due to their abundance and the low cost thereof, alginates and chitosan should be mentioned.

Chitosan is widely used in the medical sector as it shows a low immunological, pathological, and infective response.

In relation to its widespread use, chitosan has become one of the most studied polysaccharides also from a chemical point of view in order to improver the properties thereof which are useful for application purposes, in particular the viscosity and solubility thereof in water, in addition to increasing the positive charge density of the polymer or modifying the bioavailability thereof through (bio)chemical modifications.

Over recent years, various derivatives of chitosan have been obtained through chemical modifications of the polymeric chain. Usually, reactions affecting the amine residues of the glucosamine units are exploited for these modifications. In particular, the introduction of saccharide units (mono- and oligosaccharides) in a lateral chain has made it possible to obtain water-soluble chitosan derivatives.

In order to improve the physical/chemical properties, i.e. the viscosity and/or visco-elasticity, of these polysaccharides, in particular hyaluronic acid, compositions obtained from the combination of hyaluronic acid and chitosan have been also described.

Nevertheless, the polycationic nature of chitosan makes it scarcely compatible with other polysaccharides and in particular polyanionic polysaccharides and likewise hyaluronic acid. Indeed, the combination of an aqueous solution of hyaluronic acid (polyanion) and one of chitosan (polycation) leads to the instantaneous formation of insoluble coacervates. The possibility of also using polysaccharides with various charges mixed together to obtain compositions with opportune physical/chemical characteristics remains an objective to pursue given the high application interest and the multiplicity of applications in the biomedical field, but not only, of polysaccharide solutions with suitable viscosity and visco-elasticity.

Of still further interest is the possibility of obtaining mixtures of crosslinked biopolymers, with improved rheological properties.

Crosslinking increases the elastic modulus (G′) and the viscous modulus (G″) of the polysaccharide polymers.

For example, in the cosmetics field, gels with a higher G′ modulus have a greater capacity to withstand dynamic forces and are therefore useful in dermo-cosmetic uses, for example providing longer-lasting correction of nasolabial folds and marionette lines. Gels with a lower G′ modulus, meanwhile, are more suitable for areas with static and surface lines where the resistance to deformation not is a critical factor, or for the anatomical areas that require volume and softness, such as lips.

In addition to having rheological characteristics which are optimal for the desired therapeutic or cosmetic effect, the biopolymers must also be easily used, especially considering that the administration route of choice is injection. Furthermore, it is highly desirable to obtain polymers whose rheological characteristics are also favourable in the polymer production step.

### SUMMARY OF THE INVENTION

Said objects have been achieved by a polymeric composition according to claim 1.

In a further aspect, the present invention concerns a method for the preparation of said polymeric composition.

In a still further aspect, the present invention concerns the use of said polymeric composition as a biomaterial or scaffold for cell growth, preferably in the treatment of orthopaedic diseases.

In a still further aspect, the present invention concerns the use of said polymeric composition as a biomaterial or scaffold for cell growth in plastic/cosmetic surgery, haemodialysis, cardiology, angiology, ophthalmology, otolaryngology, pneumology, dentistry, gynaecology, urology, dermatology, oncology and tissue repair.

In another aspect, the present invention regards a polymeric composition further comprising at least one pharmacologically active substance and/or at least one substance with, optionally, a biological function.

In a still further aspect, the present invention concerns the use of said polymeric composition in the treatment of diseases ascribable to altered expression of galectins. Non-limiting examples of diseases concerned by over-/under-regulation of these receptors are nonalcoholic steatohepatitis, plaque psoriasis, rheumatoid arthritis, osteoarthritis, neoplasms, adhesions, and dermal, pulmonary, renal, and cardiovascular fibrotic processes. In a still further aspect, the present invention concerns the use of said polymeric composition in rheumatology, orthopaedics, oncology, plastic/cosmetic surgery, haemodialysis, cardiology, angiology, ophthalmology, otolaryngology, pneumology, dentistry, gynaecology, urology, dermatology, oncology and tissue repair.

The characteristics and the advantages of the present invention will be clear from the following detailed description and the working embodiments provided as non-limiting illustrative examples.

### DETAILED DESCRIPTION OF THE INVENTION

The invention therefore concerns a polymeric composition comprising:
a) at least one crosslinked polyanionic polysaccharide, or a crosslinked derivative thereof,
said at least one polyanionic polysaccharide, or derivative thereof, being at least partially crosslinked directly via an ester bond or a lactone bond between carboxyl groups and hydroxyl groups of the same polyanionic polysaccharide chain, or derivative thereof, and/or between carboxyl groups and hydroxyl groups of different chains, or
being at least partially indirectly crosslinked via a spacer moiety forming ester bonds with carboxyl groups and/or ether bonds with hydroxyl groups and/or amide bonds with carboxyl groups, said spacer moiety being a biscarbodiimide moiety or a bisvinylsulphonic moiety or a epoxy moiety deriving from bi- or polyfunctional epoxide selected from C2-C20 aliphatic epoxides, their halogen hydrins, epihalogen hydrins, and halides, or a combination thereof; and
b) at least one polyaminosaccharide selected from a chitosan, a functionalised chitosan, and salts thereof, wherein said functionalised chitosan is a chitosan wherein at least 20% of the D-glucosamine units has formula (I):

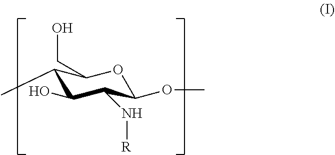

where R is a moiety of formula (1):

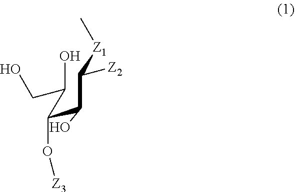

wherein Z₁ is —CH₂— or —CO—,
Z₂ is —OH, or —NHCOCH₃,
Z₃ is H, monosaccharide, disaccharide, or oligosaccharide,
or R is a moiety of formula (2):

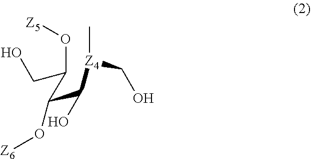

wherein Z₄ is —CH—,
Z₅ and Z₆ are, independently of each other, H, monosaccharide, disaccharide, or oligosaccharide.

The polymeric composition described above features a proper viscosity and/or visco-elasticity, so as to make said composition particularly suitable for the several known uses of polysaccharides and mixtures of polysaccharides.

In particular, said polymeric composition features improved rheological properties compared with compositions according to the prior art and in particular compared with compositions that do not comprise a chitosan polyaminosaccharide or a derivative thereof. These improved rheological properties are particularly advantageous as they make the compositions more workable for both the production thereof and the uses thereof.

In particular, these improved rheological properties are extremely advantageous for the formulation of the compositions according to the invention in aqueous form and for their administration via injection to the site of interest.

Furthermore, this composition has shown a high acceptability profile from a medical and pharmaceutical perspective, in addition to improved persistence times at the target site, since said composition features greater resistance to enzymatic degradation, in addition to improved mechanical and physical/chemical properties.

Preferably, said at least one polyanionic polysaccharide is at least one carboxylated polysaccharide or at least one sulphated polysaccharide or a mixture thereof.

More preferably, said at least one carboxylated polysaccharide is selected from hyaluronic acid, alginate, pectin, carboxymethyl cellulose, salts thereof, and mixtures thereof.

Still more preferably, said at least one carboxylate polysaccharide is hyaluronic acid or a salt thereof.

Preferably, the weight average molecular weight (Mw) of the hyaluronic acid is 10 kDa-10000 kDa, more preferably 100 kDa-5000 kDa, and still more preferably 500 kDa-3500 kDa.

More preferably, said at least one sulphated polysaccharide is selected from carrageenan, agarose sulphate, keratan sulphate, dermatan sulphate, starch sulphate, heparin, and mixtures thereof.

The carboxyl groups and the hydroxyl groups of said at least one polyanionic polysaccharide, or derivative thereof, not involved in the crosslinking can optionally be salified, for example, with cations of sodium, potassium, calcium, magnesium, ammonium or mixtures thereof.

In some embodiments, in the composition according to the invention, 20-70% of the carboxyl groups and the hydroxyl groups of said at least one polyanionic polysaccharide, or derivative thereof, not involved in the crosslinking, are salified.

Preferably, 5-40% of the bonds between the spacer moiety and said at least one polyanionic polysaccharide, or derivative thereof, are ester bonds, more preferably 10-30%.

As stated above, the crosslinking of said at least one polyanionic polysaccharide according to the invention can take place directly, i.e. by intramolecular and/or intermolecular reaction between free carboxyl and/or hydroxyl functional groups of said at least one polyanionic polysaccharide, or derivative thereof, or indirectly, i.e. by intramolecular and/or intermolecular reaction via a spacer moiety between free carboxyl and/or hydroxyl functional groups of said at least one polyanionic polysaccharide, or derivative thereof. Therefore, said at least one polyanionic polysaccharide according to the present invention can comprise the following types of direct crosslinking (wherein said at least one polyanionic polysaccharide, or derivative thereof, is denoted, for the sake of practicality, as “HYD”):

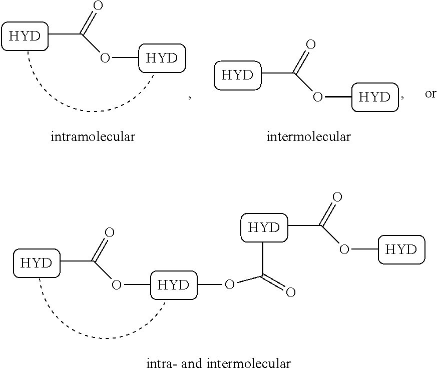

or of indirect crosslinking via a spacer moiety (denoted, for the sake of practicality, as “SPC”):

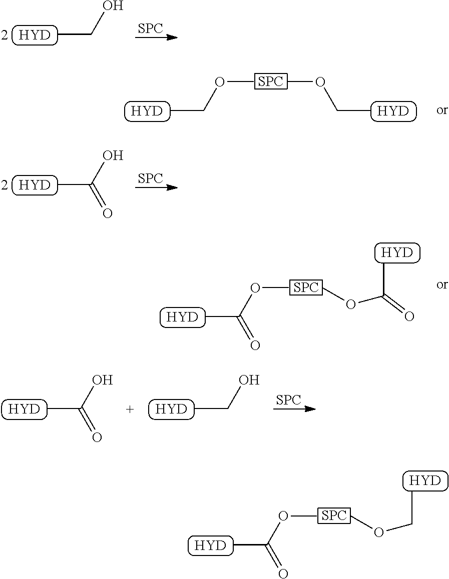

In certain embodiments, said spacer moiety is derived from bi- or polyfunctional epoxide selected from epichlorohydrin, 1,4-butanediol diglycidyl ether, 1,2-ethylene diol diglycidyl ether, 1-(2,3-epoxypropyl)-2,3-epoxycyclohexane, N,N-diglycidylaniline, epoxy-substituted pentaerythritol, and mixtures thereof.

Preferably, said spacer moiety is derived from 1,4-butanediol diglycidyl ether. In this case, the crosslinked polyanionic polysaccharide, or a crosslinked derivative thereof, according to the present invention can comprise one or more of the following types of crosslinking:

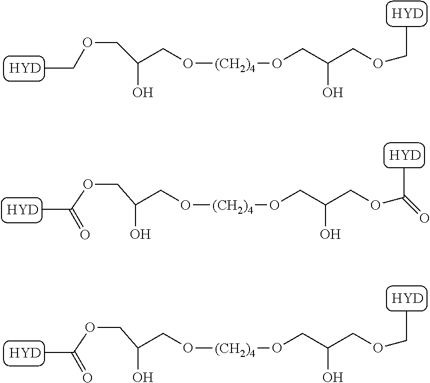

In other embodiments, said spacer moiety is derived from divinylsulphone. In this case, the crosslinked polyanionic polysaccharide, or derivative thereof, according to the present invention can comprise the following type of crosslinking:

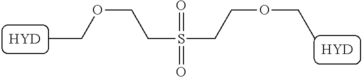

In other embodiments, said spacer moiety is derived from a biscarbodiimide of formula Y₁—N═C═N—Y₂—N═C═N—Y₃, where Y₁ and Y₃ are, independently of one another, hydrogen, linear, or branched C1-C10 aliphatic group, C1-C10 alkoxy group, C1-C10 cycloaliphatic group, C1-C10 aryl, C1-C10 heteroaryl, C1-C10 aralkyl, C1-C10 heteroaralkyl, and Y₂ is a bifunctional moiety derived from linear or branched C1-C10 aliphatic group, C1-C10 alkoxy group, C1-C10 cycloaliphatic group, C1-C10 aryl, C1-C10 heteroaryl, C1-C10 aralkyl, or C1-C10 heteroaralkyl.

In this case, said at least one polyanionic polysaccharide according to the present invention can comprise the following types of crosslinking:

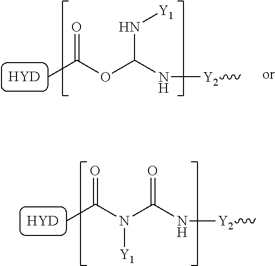

and likewise the specular types of crosslinking on the imide function of the spacer moiety comprising the substituent Y₃.

Preferably, said biscarbodiimide is selected from 1,6-hexamethylene bis(ethylcarbodiimide), 1,8-octamethylene bis(ethylcarbodiimide), 1,10 decamethylene bis(ethylcarbodiimide), 1,12 dodecamethylene bis(ethylcarbodiimide), PEG-bis(propyl (ethylcarbodiimide)), 2,2′-dithioethyl bis(ethylcarbodiimide), 1,1′-dithio-p-phenylene bis(ethylcarbodiimide), para-phenylene-bis(ethylcarbodiimide), 1,1′-dithio-m-phenylene bis(ethylcarbodiimide) and mixtures thereof.

More preferably, said biscarbodiimide is para-phenylene-bis(ethylcarbodiimide).

The term “aliphatic, aromatic, arylaliphatic, cycloaliphatic, heterocyclic” preferably means a linear, branched, or cyclic, saturated or unsaturated, aliphatic or aromatic moiety chosen from among C1-C10 alkyl, C1-C10 alkyl substituted, C2-C10 alkenyl, C2-C10 substituted alkenyl, C4-C10 dienyl, C4-C10 substituted dienyl, C2-C10 alkynyl, C2-C10 substituted alkynyl, phenyl, substituted phenyl, aryl, substituted aryl, heteroaryl, substituted heteroaryl, C1-C10 alkylthio, C1-C10 substituted alkylthio, phenylthio, substituted phenylthio, arylthio, substituted arylthio, carbonyl, C1-C6 substituted carbonyl, carboxyl, C1-C6 substituted carboxyl, amine, C1-C6 substituted amine, amide, C1-C6 substituted amide, sulphonyl, C1-C6 substituted sulphonyl, sulfonic acid, phosphonyl, C1-C6 substituted phosphonyl, polyaryl, substituted polyaryl, C3-C20 cycloalkyl, C3-C20 substituted cycloalkyl, C3-C20 heterocycloalkyl, C3-C20 substituted heterocycloalkyl, C2-C10 cycloalkenyl, C2-C10 substituted cycloalkenyl, C4-C10 cyclodienyl, C4-C10 substituted cyclodienyl, or amino acid. The term “substituted”, means bonded to at least one halogen, hydroxyl, C1-C4 alkyl, carboxyl, or combinations thereof.

As regards to at least one polyaminosaccharide b), this is selected from a chitosan, a functionalised chitosan, and salts thereof.

Preferably, said at least one polyaminosaccharide b) is functionalised chitosan, wherein up to 90% of the D-glucosamine units has formula (I), more preferably 40-80% of the D-glucosamine units has formula (I).

In some embodiments, said at least one polyaminosaccharide b) is functionalised chitosan according to formula (I) in the form of salt consisting of a cation of functionalised chitosan and a monovalent, bivalent, or trivalent anion.

Preferably, said at least one polyaminosaccharide b) is functionalised chitosan, wherein Z₃, Z₅, and Z₆ are, independently of one another, H, moiety of glucose, galactose, arabinose, xylose, mannose, lactose, trehalose, gentiobiose, cellobiose, cellotriose, maltose, maltotriose, chitobiose, chitotriose, mannobiose, melibiose, fructose, N-acetyl glucosamine, N-acetyl galactosamine, or a combination thereof.

More preferably, said at least one polyaminosaccharide b) is functionalised chitosan, wherein Z₃ is H, moiety of glucose, galactose, mannose, N-acetyl glucosamine, N-acetyl galactosamine, or a combination thereof.

In particularly preferred embodiments, said at least one polyaminosaccharide b) is functionalised chitosan, wherein R is a moiety of lactose or of galactose.

Preferably, the weight average molecular weight (Mw) of said at least one polyaminosaccharide b) is up to 2500 kDa, more preferably 250 kDa-1500 kDa, and still more preferably 400 kDa-1200 kDa.

Preferably, the numerical average molecular weight (Mn) of said at least one polyaminosaccharide b) is up to 2000 kDa, more preferably 100 kDa-1000 kDa, and still more preferably 200 kDa-600 kDa.

In a further aspect, the present invention concerns a method for the preparation of the polymeric composition according to the invention, comprising the following steps:
i) providing a) said at least one polyanionic polysaccharide, or derivative thereof;
ii) reacting said at least one polyanionic polysaccharide, or derivative thereof, with a crosslinking agent selected from biscarbodiimides, divinylsulphone, an epoxy chosen from aliphatic epoxides C2-C20, their halogenhydrons, epialogenhydrins, and halides, or methylpyridine halides, in the presence of a base, or combinations thereof, thus obtaining a crosslinked polymeric gel;
iii) adding an acid until a pH of 6.5 to 7.5 is reached;
iv) adding at least b) a polyaminosaccharide or a salt thereof; and
v) leaving the reaction at room temperature, thus obtaining the polymeric composition.

Preferably, in step ii) up to 30 wt % of the crosslinking agent is added, based on the weight of the crosslinked polymeric gel, and more preferably, up to 25 wt % of said crosslinking agent.

The crosslinkable hyaluronic acid derivatives usable in the composition according to the present invention are preferably the following:
hyaluronic acid salts, such as hyaluronate sodium, hyaluronate potassium, hyaluronate calcium, hyaluronate magnesium, hyaluronate zinc, hyaluronate cobalt, hyaluronate ammonium, hyaluronate tetrabutylammonium, and mixtures thereof,
hyaluronic acid esters wherein part or all the carboxyl groups are esterified with aliphatic, aromatic, arylaliphatic, cycloaliphatic, or heterocyclic series alcohols, as also described in EP0216453B1,
self-crosslinked hyaluronic acid esters wherein a part or all the carboxyl groups are esterified with the alcohol groups in the same polysaccharide chain or other chains, as also described in EP0341745B1,
crosslinked hyaluronic acid compounds wherein a part or all the carboxyl groups are esterified with aliphatic, aromatic, arylaliphatic, cycloaliphatic, or heterocyclic series polyalcohols, generating the crosslinkings by means of spacer chains, as also described in EP0265116B1),
succinic acid hemiesters or salts of heavy metals of succinic acid with hyaluronic acid or with partial or total hyaluronic acid esters, as also described in WO96/357207,
O-sulphated derivatives, as also described in WO95/25751, or N-sulphated derivatives, as also described in WO/1998/045335.

Preferably, the polymeric composition according to the invention is in the form of an aqueous solution or in the form of powder, more preferably is in an injectable form which is suitable for the body's hard or soft tissues, such as organs, adipose, mucosal, and gingival tissues, cartilage and bones, preferably in a form which is injectable by intradermal, subcutaneous, intramuscular, intra-articular or intraocular route.

Preferably, the polymeric composition according to the invention has a pH of 10-2, more preferably 9-4, and still more preferably 8-6.

In a further aspect, the present invention concerns the use of said polymeric composition in the treatment of diseases ascribable to altered galectin expression. Non-limiting examples of diseases concerned by over-/under-regulation of these receptors include nonalcoholic steatohepatitis, plaque psoriasis, rheumatoid arthritis, osteoarthritis, neoplasms, adhesions, and dermal, pulmonary, renal, and cardiovascular fibrotic processes.

Examples of neoplasms and fibrotic processes include acute lymphoblastic leukaemia, idiopathic pulmonary fibrosis, hepatic fibrosis, cardiac fibrosis, renal fibrosis, tumours of the ovary, of the prostate, of the lungs, of the stomach, of the skin, of the thyroid, and of the pancreas.

In another aspect, the present invention concerns the use of said polymeric composition as a biomaterial or scaffold for cell growth, preferably in the treatment of orthopaedic diseases. According to preferred aspects, the present invention concerns the use of said polymeric composition in tissue repair or reconstruction, preferably in the creation or substitution of biological tissues or in the filling of biological tissues, such as cutaneous filling, the filling of troughs, of bone cartilage or of joints.

In a still further aspect, the present invention concerns the use of said polymeric composition for cell growth, in plastic-cosmetic surgery, haemodialysis, cardiology, angiology, ophthalmology, otolaryngology, pneumology, dentistry, gynaecology, urology, dermatology, oncology and tissue repair; the present invention furthermore concerns the use of this composition in traumatic and/or post-surgical tissue processes and/or chronic fibrotic processes associated with autoimmune diseases, in traumatic and/or post-surgical sequelae involving dermal and abdominal tissues, or in post-surgical sequelae of endonasal procedures, in post-surgical sequelae of tendinous and/or cartilaginous tissues.

Particularly preferred is the use of the composition according to the invention in the treatment of asthma, COPD, IPF, tonsillitis, laryngitis, pharyngitis, nasopharyngitis, sinusitis, rhinitis, tracheitis, hoarseness of the throat, inflammation of the vocal cords with and without dysphonia.

The polymeric composition can be used also as a biomaterial for coating objects utilised both in the medical field and in other sectors of the industry, providing new a biological characteristic to the surface of the object forming the substrate.

Objects that can be coated include, for example, catheters, tubes, probes, heart valves, soft tissue prostheses, prostheses of animal origin, artificial tendons, bone and cardiovascular prostheses, contact lenses, oxygenators for blood, kidneys, heart, pancreas, artificial livers, blood bags, syringes, surgical instruments, filtration systems, laboratory instruments, containers, for cultures and for the regeneration of cells and tissues, substrates for peptides, proteins, and antibodies.

The polymeric composition can be used also in the cosmetic and dermatological fields in dermatological or cosmetic products, or as a biomedical product, preferably as a bioresorbable implant.

In a still further aspect, the present invention concerns the use of said polymeric composition in psoriasis and in psoriatic osteoarthritis.

Preferably, the polymeric composition further comprises at least one pharmacologically active substance and/or at least one substance with, optionally, a biological function. Suitable pharmacologically active substances include antibiotic, anti-infective, anti-microbial, antiviral, cytostatic, cytotoxic, antitumour, anti-inflammatory, healing, anaesthetic, analgesic, vasoconstriction, cholinergic or adrenergic agonistic and antagonistic, antithrombotic, anticoagulant, haemostatic, fibrinolytic, and thrombolytic agents, as well as proteins and fragments thereof, peptides, polynucleotides, growth factors, enzymes, vaccines, or combinations thereof.

Preferably, said substance with—optionally—a biological function is chosen from among collagen, fibrinogen, fibrin, alginic acid, sodium alginate, potassium alginate, magnesium alginate, cellulose, chondroitin sulphate, dermatan sulphate, keratan sulphate, heparin, heparan sulphate, laminin, fibronectin, elastin, polylactic acid, polyglycolic acid, acid poly(lactic-co-glycolic), polycaprolactone, gelatin, albumin, poly(glycolide-co-caprolactone), poly(glycolide-co-trimethylene carbonate), hydroxyapatite, tricalcium phosphate, dicalcium phosphate, demineralised bone matrix, and mixtures thereof.

Preferably, the polymeric composition and said at least one substance with—optionally—a biological function have a weight ratio ranging from 100:1 to 1:150.

Preferably, the composition according to the invention comprises up to 10 wt % of said at least one crosslinked polyanionic polysaccharide, or derivative thereof, based on the weight of the polymeric composition, more preferably, up to 5 wt % of said at least one crosslinked polyanionic polysaccharide, or derivative thereof. Particularly preferred are pharmaceutical compositions wherein said at least one crosslinked polyanionic polysaccharide, or derivative thereof, amounts to 0.1-5 wt %, based on the weight of the composition.

Preferably said crosslinked polyanionic polysaccharide, or derivative thereof, and said polyaminosaccharide are present in the polymeric composition with a weight ratio ranging from 1:10 to 10:1, more preferably from 5:1 to 1:5, and still more preferably from 5:1 to 1:2.

The composition according to the invention can further comprise pharmaceutically acceptable excipients.

Suitable pharmaceutically acceptable excipients are, for example, pH regulators, isotonic regulators, solvents, stabilisers, chelating agents, diluting agents, binding agents, disintegrating agents, lubricating agents, glidants, colouring agents, suspending agents, surfactants, cryoprotectants, preservatives, and antioxidants.

The present invention furthermore concerns a biomaterial comprising the polymeric composition according to the invention alone or in combination with at least one of the pharmacologically active and/or bioactive substances described above. Said biomaterial can be in the form of microspheres, nanospheres, membrane, sponge, thread, film, gauze, guide channel, swab, gel, hydrogel, fabric, non-woven fabric, tube, or a combination thereof.

It should be understood that the aspects identified as preferred and advantageous for the polymeric composition should likewise be considered preferred and advantageous also for the method of preparation, the compositions, the biomaterials and the uses stated above.

It should furthermore be understood that all the possible combinations of the preferred aspects according to the invention stated above have also been described and are therefore likewise preferred.

Below are working examples of the present invention provided for illustrative purposes.

### EXAMPLES

### Example 1

### Preparation of Functionalised Chitosan (Chitlac)

Lactose (36 g), water (500 mL), acetic acid (100%) and chitosan (12 g) were loaded into a reactor and the mixture thus obtained was heated to 60° C. for 2 hours. Next, in the same conditions, 2-methylpyridine borane (8 g) previously dispersed in methanol (80 mL) was added gradually and the system was left under stirring at 60° C. for 2 hours. Subsequently, an aqueous solution of hydrochloric acid (4 N) was added dropwise until a pH value of approximately 2 was reached. Next, the system was cooled to room temperature and the product was precipitated by adding 2-propanol. Subsequently, the precipitate was decanted, the supernatant removed, and the solid reside washed a first time with a mixture of water:2-propanol (30:70), several times with mixtures of water:2-propanol (15:85), and a final time with 2-propanol. The solid was then dried under low pressure and controlled temperature conditions.

### Example 2

Preparation of a Mixture of 20 mg/mL Crosslinked Hyaluronic Acid (HY) and 5 mg/mL Chitlac with 5% butanediol-diglycidyl-ether (BDDE)

27.3 μL BDDE (5% with respect to the hyaluronic acid) was added to 8 mL NaOH 0.25 M and the solution was kept under stirring for 5 min until homogenisation was achieved. 1.2 g hyaluronic acid was weighed into a beaker, to which the NaOH-BDDE mixture prepared previously was added. The solution was kept under stirring until complete solubilisation of the hyaluronic acid. The reaction mixture was then kept at 45° ° C. for 4 hours to make the BDDE crosslink. At the same time, 0.3 g Chitlac was dissolved in 15 mL injectable water in a round-bottom flask. Once the Chitlac was solubilised, 6 mL PBS 10× was added and the pH was taken to 7 with NaOH 0.25 M.

At the end of the 4-hour reaction time, the beaker was cooled to room temperature and the gel was crushed. 5 mL injectable water and 5 mL HCl 0.1 M were then added and the gel was left to rest for 10 min until complete absorption of the solvent. The pH was then taken to 7 by adding small aliquots of HCl 0.1 M, crushing the product and then leaving it to rest between one addition and the next. Once the gel was neutralised, the Chitlac solution was added to the beaker and mixed until homogeneous. Then it was brought up to volume with injectable water and, after mixing, was left to incubate at room temperature for 16 hours with stirring switched off. The product was then subjected to dialysis for 2 days in PBS 1× with membrane with cut-off of 10,000 Da. Finally, the gel was divided into syringes, from which the air was removed by vacuum, and the product was sterilised in autoclave at 121° C. for 15 min.

The same process was also carried out without the dialysis step.

### Example 3

Preparation of a mixture of 20 mg/mL crosslinked hyaluronic acid crosslinked (HY) and 5 mg/mL Chitlac with 10% butanediol-diglycidyl-ether (BDDE)

54.6 μL BDDE (10% with respect to the hyaluronic acid) was added to 8 mL of NaOH 0.25 M and the solution was kept under stirring for 5 min until homogeneous. 1.2 g hyaluronic acid was weighed into a beaker, to which the NaOH-BDDE mixture prepared previously was added. The solution was kept under stirring until complete solubilisation of the hyaluronic acid. The reaction mixture was then kept at 45° C. for 4 hours to allow the BDDE to crosslink. At the same time, 0.3 g Chitlac was dissolved in 15 mL injectable water in a round-bottom flask. Once the Chitlac was solubilised, 6 mL PBS 10× was added and the pH was taken to 7 with NaOH 0.25 M.

At the end of the 4-hour reaction time, the beaker was cooled to room temperature and the gel was crushed. 5 mL injectable water and 5 mL HCl 0.1 M were then added and the gel was left to rest for 10 min until complete absorption of the solvent. The pH was then taken to 7 by adding small aliquots of HCl 0.1 M, crushing the product and then leaving it to rest between one addition and the next. Once the gel was neutralised, the Chitlac solution was added to the beaker and mixed until homogeneous. It was then brought up to volume (60 mL) with injectable water and, after mixing, was left to incubate at room temperature for 16 hours with stirring switched off. The product was then subjected to dialysis for 2 days in PBS 1× with membrane with cut-off of 10.000 Da. Finally, the gel was divided into syringes, from which the air was removed by vacuum and the product was sterilised in autoclave at 121ºC for 15 min.

The same process was also carried out without the dialysis step.

### Example 4

Preparation of a Mixture of Crosslinked 20 mg/mL HY and 5 mg/ml Chitlac with 12.5% BDDE

68.3 μL BDDE (10% with respect to the hyaluronic acid) was added to 8 mL of NaOH 0.25 M and the solution was kept under stirring for 5 min until homogeneous. 1,2 g hyaluronic acid was weighed into a beaker, to which the NaOH-BDDE mixture prepared previously was added. The solution was kept under stirring until complete solubilisation of the hyaluronic acid. The reaction mixture was then kept at 45ºC for 4 hours to allow the BDDE to crosslink. At the same time, 0.3 g Chitlac was dissolved in 15 mL injectable water in a round-bottom flask. Once the Chitlac was solubilised, 6 mL PBS 10× was added and the pH was taken to 7 with NaOH 0.25 M.

At the end of the 4-hour reaction time, the beaker was cooled to room temperature and the gel was crushed. 5 mL injectable water and 5 mL HCl 0.1 M were then added and the gel was left to rest for 10 min until complete absorption of the solvent. The pH was then taken to 7 by adding small aliquots of HCl 0.1 M, crushing the product and then leaving it to rest between one addition and the next. Once the gel was neutralised, the Chitlac solution was added to the beaker and mixed until homogeneous. It was then brought up to volume (60 mL) with injectable water and, after mixing, was left to incubate at room temperature for 16 hours with stirring switched off. The product was then subjected to dialysis for 2 days in PBS 1× with membrane with cut-off of 10.000 Da. Finally, the gel was divided into syringes, from which the air was removed by vacuum and the product was sterilised in autoclave at 121ºC for 15 min.

The same process was also carried out without the dialysis step.

### Example 5

Preparation of 20 mg/mL Crosslinked HY with 5% BDDE.

27.3 μL of BDDE (5% with respect to the hyaluronic acid) was added to 8 mL of NaOH 0.25 M and the solution was kept under stirring for 5 min until homogeneous. 1.2 g hyaluronic acid was weighed into a beaker, to which the NaOH-BDDE mixture prepared previously was added. The solution was kept under stirring until complete solubilisation of the hyaluronic acid. The reaction mixture was then kept at 45ºC for 4 hours to allow the BDDE to crosslink.

At the end of the 4-hour reaction time, the beaker was cooled to room temperature and the gel was crushed. 5 mL injectable water and 5 mL HCl 0.1 M were then added and the gel was left to rest for 10 min until complete absorption of the solvent. The pH was then taken to 7 by adding small aliquots of HCl 0,1 M, crushing the product and then leaving it to rest between one addition and the next. Once the gel was neutralised, 6 mL PBS 10× was added and mixed until homogeneous. It was then brought up to volume with injectable water and, after mixing, was left to incubate at room temperature for 16 hours with stirring switched off. The product was then subjected to dialysis for 2 days in PBS 1× with membrane cut-off of 10000 Da. Finally, the gel was divided into syringes, from which the air was removed by vacuum and the product was sterilised in autoclave at 121ºC for 15 min.

The same process was also carried out without the dialysis step.

### Example 6

Preparation of HY 20 mg/mL Crosslinked with 10% BDDE

68.3 μL BDDE (10% with respect to the hyaluronic acid) was added to 8 mL of NaOH 0.25 M and the solution was kept under stirring for 5 min until homogeneous. 1.2 g hyaluronic acid was weighed into a beaker, to which the NaOH-BDDE mixture prepared previously was added. The solution was kept under stirring until complete solubilisation of the hyaluronic acid. The reaction mixture was then kept at 45ºC for 4 hours to allow the BDDE to crosslink.

At the end of the 4-hour reaction time, the beaker was cooled to room temperature and the gel was crushed. 5 mL injectable water was then added and the gel was left to rest for 10 min until complete absorption of the solvent. The pH was then taken to 7 by adding small aliquots of HCl 0,1 M, crushing the product and then leaving it to rest between one addition and the next. Once the gel was neutralised, 6 mL PBS 10× was added and mixed until homogeneous. It was then brought up to volume (60 mL) with injectable water and, after mixing, was left to incubate at room temperature for 16 hours with stirring switched off. The product was then subjected to dialysis for 2 days in PBS 1× with membrane with cut-off of 10.000 Da. Finally, the gel was divided into syringes, from which the air was removed by vacuum and the product was sterilised in autoclave at 121ºC for 15 min. The same process was also carried out without the dialysis step.

### Example 7

Preparation of 20 mg/mL Crosslinked HY with 12.5% BDDE

54.6 μL BDDE (12.5% with respect to the hyaluronic acid) was added to 8 mL of NaOH 0.25 M and the solution was kept under stirring for 5 min until homogeneous. 1.2 g hyaluronic acid was weighed into a beaker, to which the NaOH-BDDE mixture prepared previously was added. The solution was kept under stirring until complete solubilisation of the hyaluronic acid. The reaction mixture was then kept at 45ºC for 4 hours to allow the BDDE to crosslink.

At the end of the 4-hour reaction time, the beaker was cooled to room temperature and the gel was crushed. 5 mL injectable water was then added and the gel was left to rest for 10 min until complete absorption of the solvent. The pH was then taken to 7 by adding small aliquots of HCl 0.1 M, crushing the product and then leaving it to rest between one addition and the next. Once the gel was neutralised, 6 mL PBS 10× was added and mixed until homogeneous. It was then brought up to volume (60 mL) with injectable water and, after mixing, was left to incubate at room temperature for 16 hours with stirring switched off. The product was then subjected to dialysis for 2 days in PBS 1× with membrane with cut-off of 10000 Da. Finally, the gel was divided into syringes, from which the air was removed by vacuum and the product was sterilised in autoclave at 121ºC for 15 min. The same process was also carried out without the dialysis step.

### Example 8

The Rheological Properties of the Compositions in Examples 3 and 6 Described Above were Measured.

The rheological analysis was conducted with the Kinexus lab+ rheometer (Malvern Instruments) using a parallel plate arrangement with a plate diameter of 2 cm and thermostatting at 20° C. To measure visco-elasticity frequency sweep measurements were taken with a 0.5 mm gap and applying a stress of 5 Pa from 0.01 to 10 Hz, 10 points/decade. The percentage of elasticity was then calculated with the formula: Elasticity (%)=(G′*100)/(G′+G″).

| | G′ (Pa) | | | | G″ (Pa) | | | | Elasticity (%) | | | |
|---|---|---|---|---|---|---|---|---|---|---|---|---|
| Sample | 0.1 Hz | 0.5 Hz | 1 Hz | 2.5 Hz | 0.1 Hz | 0.5 Hz | 1 Hz | 2.5 Hz | 0.1 Hz | 0.5 Hz | 1 Hz | 2.5 Hz |
| 20 mg/mL HY, | 72.6 | 83.1 | 90.7 | 113.8 | 8.5 | 12.9 | 16.0 | 22.7 | 89.6 | 86.6 | 85.0 | 83.4 |
| sterile 10% | | | | | | | | | | | | |
| BDDE | | | | | | | | | | | | |
| 20 mg/mL HY, | 197 | 211 | 222 | 254 | 11.7 | 19.3 | 24.3 | 33.5 | 94.4 | 91.6 | 90.1 | 88.3 |
| CTL 5 mg/mL, | | | | | | | | | | | | |
| sterile 10% | | | | | | | | | | | | |
| BDDE | | | | | | | | | | | | |

It is clear that the addition of the functionalised chitosan to the composition comprising crosslinked hyaluronic acid determines a high increase in the G′ and G″ moduli, in particular the G′ modulus, with consequent improvement of the elasticity rate.

1. A polymeric composition comprising:
a) at least one crosslinked polyanionic polysaccharide, or a crosslinked derivative thereof, said polyanionic polysaccharide, or derivative thereof,
said polyanionic polysaccharide, or derivative thereof, being at least partially crosslinked directly via ester bond or lactone bond between carboxyl groups and hydroxyl groups of the same chain of polyanionic polysaccharide, and/or between carboxyl groups and hydroxyl groups of different chains, or
being at least partially crosslinked indirectly via a spacer moiety forming ester bonds with the carboxylic groups and/or ether bonds with the hydroxyl groups and/or amide bonds with the carboxyl groups, said spacer moiety being a biscarbodiimidic moiety or a bisvinylsulfonic moiety or an epoxy moiety deriving from bi- or polyfunctional epoxide selected from C2-C20 aliphatic epoxides, their halogenhydrons, epialogenhydrins, and halides, or a combination thereof; and
b) at least one polyamine-saccharide, or a derivative thereof, selected from a chitosan, a functionalized chitosan, and salts thereof, wherein said functionalized chitosan is a chitosan wherein at least 20% of D-glucosamine units is of formula (I): 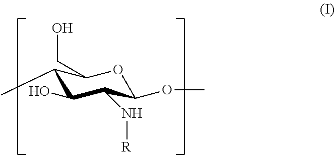
wherein R is a moiety of formula (1): 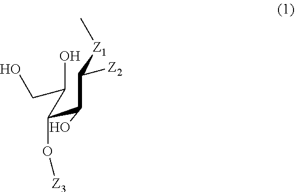
wherein Z₁ is —CH₂— or [o]—CO—,
Z₂ is —OH, or —NHCOCH₃,
Z₃ is H, monosaccharide, disaccharide, or oligosaccharide,
or R is a moiety of formula (2): 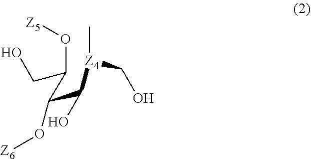
wherein Z₄ is —CH—,
Z₅ and Z₆ are, independently of each other, H, monosaccharide, disaccharide, or oligosaccharide.

2. The composition of claim 1, wherein a) at least one polyanionic polysaccharide is at least one carboxylated polysaccharide or at least one sulphated polysaccharide or mixtures thereof.

3. The composition of claim 2, wherein said a) at least one carboxylated polysaccharide is selected from hyaluronic acid, alginate, pectin, carboxymethylcellulose, their salts, and their mixtures.

4. The composition of claim 3, wherein said a) at least one carboxylated polysaccharide is hyaluronic acid, or salt thereof.

5. The composition of claim 4, wherein the weight average molecular weight (Mw) of hyaluronic acid is 10 kDa-10000 kDa.

6. The composition of claim 2, wherein said a) at least one sulphated polysaccharide is selected from carrageenan, agarose sulphate, keratan sulphate, dermatan sulphate, starch sulphate, heparin, and their mixtures.

7. The composition of claim 1, wherein said b) at least one polyamine-saccharide is functionalized chitosan, wherein 40-80% of D-glucosamine units have formula (I).

8. The composition of claim 7, wherein said b) at least one polyamine-saccharide is functionalized chitosan of formula (I) in the form of a salt consisting of functionalized chitosan cation and monovalent, bivalent or trivalent anion.

9. The composition of claim 1, wherein said b) at least one polyamine-saccharide is functionalized chitosan of formula (I), wherein Z₃, Z₅ e Z₆ are, independently of one another, H, a moiety of glucose, galactose, arabinose, xylose, mannose, lactose, trehalose, gentiobiose, cellobiose, cellotriose, maltose, maltotriose, chitobiose, chitotriose, mannobiose, melibiose, fructose, N-acetyl glucosamine, N-acetyl galactosamine, or a combination thereof.

10. The composition of claim 9, wherein said b) at least one polyamine-saccharide is functionalized chitosan of formula (I), wherein R is a moiety of lactose or galactose.

11. The composition of claim 1, wherein:
the weight average molecular weight (Mw) of said b) at least one polyamine-saccharide is up to 2500 kDa; or
the number average molecular weight (Mn) of said b) at least one polyamine-saccharide is up to 2000 kDa.

12. A process for preparing the composition of claim 1, comprising the steps of:
i) providing said a) at least one polyanionic polysaccharide, or a derivative thereof;
ii) reacting said polyanionic polysaccharide, or a derivative thereof, with a crosslinking agent selected from biscarbodiimides, or divinilsulfone, or an epoxy compound selected from C2-C20 aliphatic epoxides, their halogenhydrons, epialogenhydrins, and halides, or methylpyridinium halides in the presence of a base, or a combination thereof, obtaining a crosslinked polymer gel;
iii) adding an acid down to a pH between 6.5 and 7.5;
iv) adding b) at least one polyamine-saccharide or a salt thereof; and
v) leaving the reaction mixture at room temperature, obtaining the polymeric composition.

13. The composition of claim 1, further comprising at least one pharmacologically active substance and/or at least one substance optionally having a biological function, wherein:
said pharmacologically active substance is selected from antibiotics, anti-infectives, antimicrobials, antivirals, cytostatic, cytotoxic, antitumor, anti-inflammatory, cicatrizing, anesthetics, analgesics, vasoconstrictors, cholinergic or adrenergic agonists and antagonists, antithrombotic, anticoagulant, hemostatic, fibrinolytic, thrombolytic, proteins and fragments thereof, peptides, polynucleotides, growth factors, enzymes, vaccines, and combinations thereof, and
said substance optionally having a biological function is selected from collagen, fibrinogen, fibrin, alginic acid, sodium alginate, potassium alginate, magnesium alginate, cellulose, chondroitin sulfate, dermatan sulfate, keratan sulfate, heparin, heparan sulfate, laminin, fibronectin, elastin, polylactic acid, polyglycolic acid, poly(lactic-co-glycolic acid), polycaprolactone, gelatin, albumin, poly(glycolide-co-caprolactone), poly(glycolide-co-trimethylene carbonate), hydroxyapatite, tricalcium phosphate, dicalcium phosphate, demineralized bone matrix, and mixtures thereof.

14. The composition of claim 1, having pH of 10-2.

15. The composition of claim 1, in an injectable form suitable for hard or soft tissues of the body, said hard or soft tissues comprising organs, adipose tissues, mucous membranes, gums, cartilage and bones, via intradermal, subcutaneous, intramuscular, intra-articular or intraocular route.

16. The composition of claim 1, in the form of an aqueous solution or powder.

17. A method for treating a subject suffering from pathologies ascribable to an altered expression of galectins, said pathologies comprising nonalcoholic steatohepatitis, plaque psoriasis, rheumatoid arthritis, osteoarthritis, neoplasia, adhesions, and dermal, pulmonary, renal, and cardiovascular fibrotic processes, the method comprising
administering an effective amount of the composition of claim 1 to the subject in need thereof.

18. The composition of claim 1 in the form of a biomaterial or scaffold for cell growth.

19.-24. (canceled)
